(19) United States
(12) Patent Application Publication
(10) Pub. No.: US 2005/0095628 A1
(43) Pub. Date: May 5, 2005

(54) Program for regulating health conditions

(76) Inventors: David W. Krempin, Temecula, CA (US); Kenneth S. Kornman, Newton, MA (US); Russell K. Randolph, Anaheim, CA (US); Haeri Roh-Schmidt, Stockton, CA (US)

Correspondence Address: ALTICOR INC., 7575 FULTON STREET EAST MAILCODE 78-2G, ADA, MI, 49355, US

(21) Appl. No.: 10/938,322

(22) Filed: Sep. 10, 2004

Related U.S. Application Data
(60) Provisional application No. 60/502,807, filed on Sep. 12, 2003.

(51) Int. Cl. C12Q 1/68

(52) U.S. Cl. 435/6

(57) ABSTRACT

A program for regulating a health condition. The program includes one or more assessments including a genetic test, biomarker test, and lifestyle assessment; a personalized intervention; and a follow up test for monitoring a subject's health condition.

This application claims the benefit of U.S. Provisional Application Ser. No. 60/502,807, filed Sep. 12, 2003.

### BACKGROUND OF THE INVENTION

The present invention relates to a program for regulating health conditions in a subject through health assessments, personalized interventions, and monitoring health. Personalized programs and products in the field of nutrition, skin care, hair care, and weight management are becoming increasingly popular in the marketplace. The basis for this includes the observation that individuals do not benefit equally, or at all, from a “one size fits all” solution. Emerging research demonstrates that at least part of individualized responsiveness to interventions is due to several differences including lifestyle, diet, and genetic makeup. As individuals define themselves as unique, and as advancements in science support individuality, the “one size fits all” model is rapidly becoming out dated. Accordingly, there remains a need to provide improved programs to assess health conditions and provide personalized interventions. Additionally, a need exists to assess the effectiveness of the personalized interventions through tracking an individual's response to the personalized interventions, thereby, monitoring the health condition.

### BRIEF DESCRIPTION OF THE DRAWINGS

FIG. 1 is a flow chart showing the general aspects of the present invention.

FIGS. 2A and 2B are flow charts outlining one embodiment of the present invention driven by an exemplary web site.

FIG. 3 is a sample results report for an individual who has completed the nutrition and lifestyle assessment and biomarker test.

FIG. 4 is a flow chart showing the secure transfer of information related to the genetic testing kits and the biomarker kits.

FIGS. 5A and 5B shows sample web pages outlining a personalized intervention recommendation.

FIG. 6 shows a sample report of the tracking feature for the present invention.

### SUMMARY OF THE INVENTION

The present invention is directed to a program for regulating health conditions in a subject comprising providing a genetic test for determining a subject's susceptibility or predisposition to a health condition; selecting and administering a personalized intervention for regulating the health condition; and monitoring the health condition.

In one embodiment, the present invention is directed to a program for regulating an inflammatory condition associated with a genetic predisposition to over-expression or altered biological activity of IL-1 in a subject. Exemplary inflammatory conditions for the present invention include cardiovascular diseases, osteoporosis, obesity, skin-related conditions, and hair-related conditions.

In accordance with one aspect of the invention, the program includes a nutrition and lifestyle assessment.

In accordance with another aspect of the present invention, the monitoring step includes a biomarker test for measuring a biomarker associated with a health condition.

In accordance with yet another aspect of the invention, the program includes education and counseling.

In accordance with yet another aspect of the present invention, the program includes a secure database for storing results of the genetic test, biomarker test, or nutrition and lifestyle assessment.

In accordance with yet another aspect of the present invention, the program includes a personalized web portal for facilitating access to one or more of the following: health assessments, education, counseling, personalized interventions, and monitoring tools.

These and other objects, advantages, and features of the invention will be better understood by reference to the drawings and the detailed description of the preferred embodiment.

### DETAILED DESCRIPTION OF THE PREFERRED EMBODIMENT

The phrase “health condition” or “health conditions” refers to a wide variety of conditions and lifestyles that can be altered by an intervention. Non-limiting examples include hair related conditions such as alopecia or thinning of the hair, natural color loss or greying, elasticity, and shine; skin related conditions such as hyperpigmentation, skin texture (smoothness), eczema, rosacea, flexibility, facial wrinkles and fine lines and associated conditions such as collagen cross-linking and collagen degradation, firmness, moisture retention, psoriasis, acne, scarring, and warts; muscle density and endurance for sports performance; and obesity and weight-related conditions. Additional examples include inflammatory or degenerative diseases including Systemic Inflammatory Response (SIRS); Alzheimer's Disease and associated conditions and symptoms including chronic neuroinflammation, glial activation, increased microglia, neuritic plaque formation, and response to therapy; amylotropic lateral sclerosis (ALS); arthritis and associated conditions and symptoms including acute joint inflammation, antigen-induced arthritis, arthritis associated with chronic lymphocytic thyroiditis, collagen-induced arthritis, juvenile chronic arthritis, juvenile rheumatoid arthritis, osteoarthritis, prognosis and streptococcus-induced arthritis; asthma and associated conditions and symptoms including bronchial asthma, chronic obstructive airway disease, chronic obstructive pulmonary disease, juvenile asthma and occupational asthma; cardiovascular diseases and associated conditions and symptoms including atherosclerosis, autoimmune myocarditis, chronic cardiac hypoxia, congestive heart failure, coronary artery disease, cardiomyopathy and cardiac cell dysfunction including aortic smooth muscle cell activation, cardiac cell apoptosis, and immunomodulation of cardiac cell function; diabetes and associated conditions and symptoms including autoimmune diabetes, insulin-dependent (Type 1) diabetes, diabetic periodontitis, diabetic retinopathy, and diabetic nephropathy; gastrointestinal inflammations and related conditions and symptoms, including celiac disease, associated osteopenia, chronic colitis, Crohn's disease, inflammatory bowel disease and ulcerative colitis; gastric ulcers; hepatic inflammations; cholesterol gallstones; and hepatic fibrosis; HIV infection and associated conditions and symptoms including degenerative responses, neurodegenerative responses, and HIV associated Hodgkin's disease; Kawasaki's syndrome and associated diseases and conditions including mucocutaneous lymph node syndrome, cervical lymphadenopathy, coronary artery lesions, edema, fever, increased leukocytes, mild anemia, skin peeling, rash, conjunctiva redness, thrombocytosis; multiple sclerosis; nephropathies and associated diseases and conditions, including diabetic nephropathy, endstage renal disease, glomerulonephritis, Goodpasture's syndrome, hemodialysis survival and renal ischemic reperfusion injury; neurodegenerative diseases and associated diseases and conditions including acute neurodegeneration, induction of interleukin-1 in aging and neurodegenerative disease, interleukin-1 induced plasticity of hypothalamic neurons and chronic stress hyperresponsiveness; ophthalmopathies and associated diseases and conditions including diabetic retinopathy, Graves ophthalmopathy, and uveitis; osteoporosis and associated diseases and conditions including alveolar, femoral, radial, vertebral or wrist bone loss or fracture incidence, postmenopausal bone loss, mass, fracture incidence or rate of bone loss; otitis media (adult or pediatric); pancreatitis or pancreatic acinitis; periodontal disease and associated diseases and conditions including adult early onset and diabetic; pulmonary diseases including chronic lung disease, chronic sinusitis, hyaline membrane disease, hypoxia and pulmonary disease in SIDS; restenosis; rheumatism including rheumatoid arthritis, rheumatic aschoff bodies, rheumatic diseases and rheumatic myocarditis; thyroiditis including chronic lymphocytic thyroiditis; urinary tract infections including chronic prostatitis, chronic pelvic pain syndrome and urolithiasis. Additional examples include immunological disorders including autoimmune diseases, such as autoimmune myocarditis, Graves' diseases, lichen sclerosis, systemic lupus erythematosus, systemic sclerosis, thyroid diseases (e.g. goiter and struma lymphomatosa, Hashimoto's thyroiditis, lymphadenoid goiter), sleep disorders, and chronic fatigue syndrome; resistance to infectious diseases, such as Leishmaniasis, Leprosy, lyme disease, lyme carditis, malaria, cerebral malaria, meningitis, tubulointestinal nephritis associated with malaris which are caused by bacteria, viruses (e.g. cytomegalovirus, encephalitis, Epstein-Barr virus, human immunodeficieny virus, influenza virus) or protozoans (e.g., Plasmodium falciparum, trypanosomes); response to trauma, including cerebral trauma (including strokes and ischemias, encephalitis, encephalopathies, epilepsy, perinatal brain injury, prolonged febrile seizures, SIDS and subarachnoid hemorrhage); low birth weight (e.g. cerebral palsy); lung injury (acute hemorrhagic lung injury, Good-Pasture's syndrome, acute ischemic reperfusion); myocardial dysfunction caused by occupational and environmental pollutants (e.g. susceptibility to toxic oil syndrome silicosis); radiation trauma; and efficiency of wound healing responses (e.g. burn or thermal wounds, chronic wounds, surgical wounds and spinal cord injuries); susceptibility to neoplasias including breast cancer associated osteolytic metastasis, cachexia, colorectal cancer, hyperproliferative diseases, Hodgkin's disease, leukemias, lymphomas, metabolic diseases and tumors, metastases, myelomas, and various cancers (including breast prostate ovarian, colon, lung, etc), anorexia and cachexia; hormonal regulation including fertility/fecundity, likelihood of a pregnancy, incidence of preterm labor, prenatal and neonatal complications including preterm low birth weight, cerebral palsy, septicemia, hypothyroxinernia, oxygen dependence, cranial abnormality, early onset menopause; a subject's response to transplant (rejection or acceptance); acute phase response (e.g. febrile response); general inflammatory response; acute respiratory distress response; acute systemic inflammatory response; wound healing; adhesion; immunoinflammatory response; neuroendocrine response; fever development and resistance; stress response; disease susceptibility; repetitive motion stress; tennis elbow; and pain management and response.

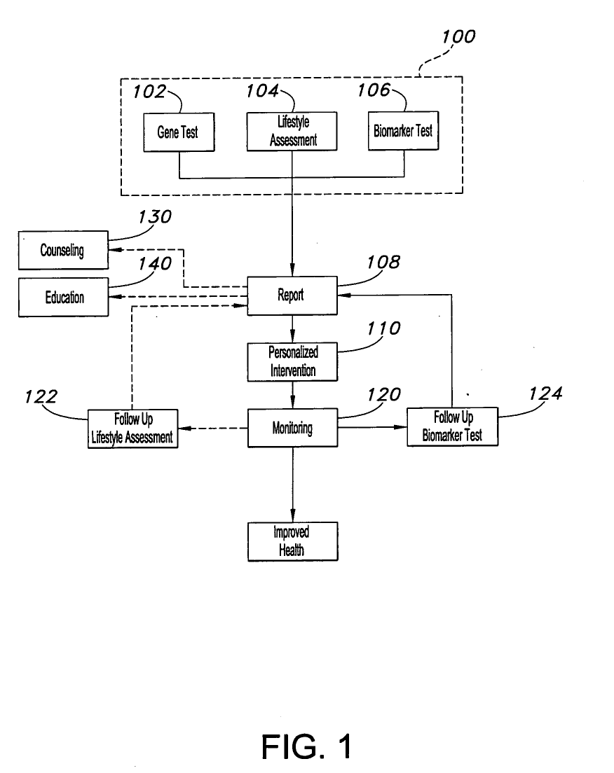

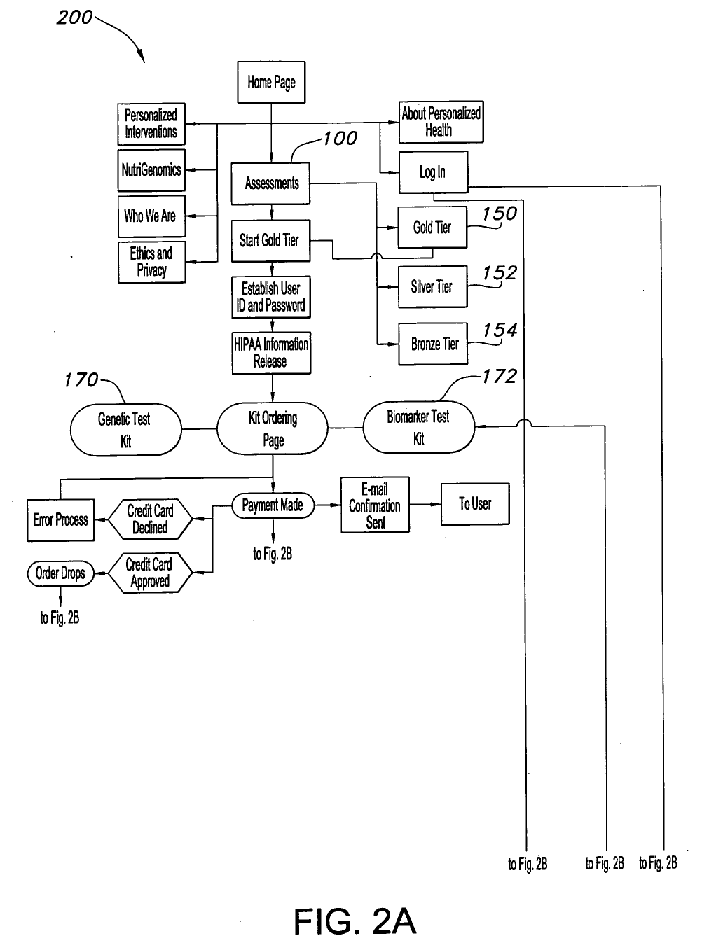

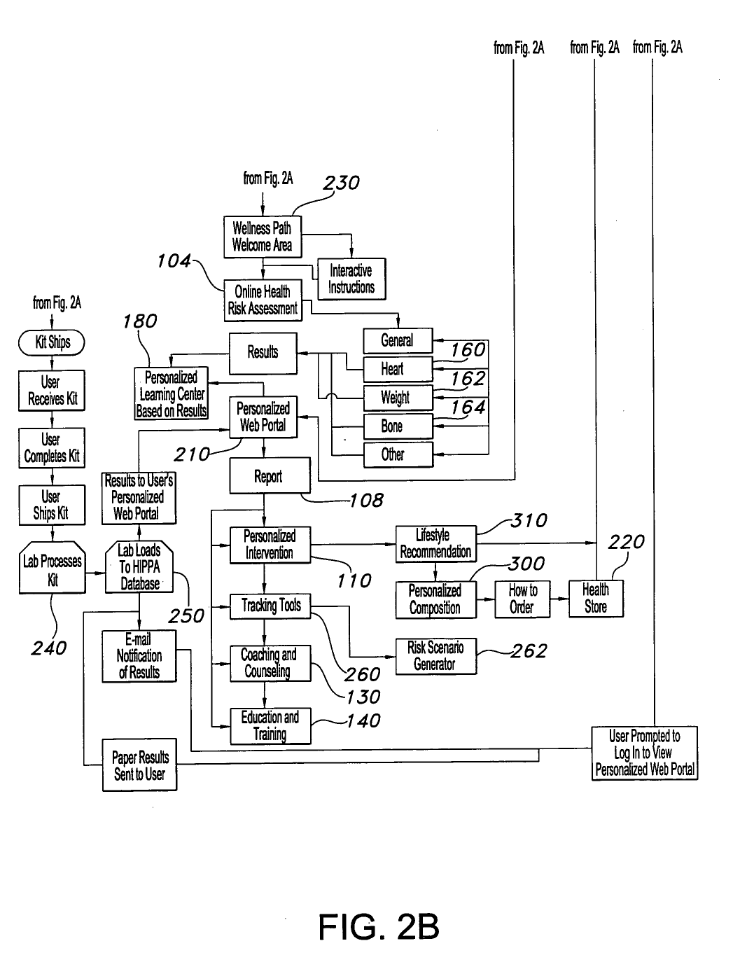

FIG. 1 shows a flow chart outlining general aspects of the present invention. Through the use of one or more health assessments 100, a personalized intervention 110, and monitoring health 120, the program of the present invention can help support healthy conditions. Counseling 130 and education 140 further support an individual's health goals. In a preferred embodiment, the aforementioned aspects of the present invention are driven by a computer assisted program, network, or web site. FIG. 2 shows a flow chart outlining one embodiment of the present invention driven by an exemplary website 200.

Assessments

Referring to FIG. 1, 2A and 2B, the program of the present invention may begin with the offering of one or more assessments 100, which can be utilized as tools to help select personalized interventions 110 for subjects. The assessments 100 include: (1) a nutrition and lifestyle assessment (“NLA”) 104; (2) a genetic test 102 to assess gene variations that are associated with certain health conditions; and (3) a biomarker test 106 for detecting and measuring biomarkers levels associated with the health condition. Any assessment 100 can be utilized separately or in combination with other assessment tools. As shown in FIG. 2A, one embodiment of the present invention bundles the assessments 100 into three tiers. The bronze tier 154 provides only a NLA 104 based on answers to a comprehensive health questionnaire. The silver tier 152 offers a more comprehensive assessment that builds on the bronze tier 154 with an evaluation of specific biomarkers for evidence of certain health risks. The gold tier 150 offers the most thorough assessment based on an individual's specific health risks. It incorporates all of the elements of the silver tier 152, plus a genetic test 102 completed in the privacy of the subject's home. FIG. 2A provides an exemplary flow chart for the gold tier 150 of the present invention.

The NLA 104 may be available as both a paper assessment and an interactive computer assisted assessment. The NLA 104 is a questionnaire that covers the state of a individual's overall health, physical activity habits, medical history, personal characteristics, and readiness to change. The NLA 104 can also identify and further discern specific health areas of interest to the individual. In this regard, the NLA 104 is modular. The modules include heart health 160, weight management 162, and bone health 164. It also may include brain health, children's health, digestive health, emotional health, energy, free radical fighters, immune health, joint health, liver health, men's health, sports nutrition, vision, and women's health. In the modular embodiment, the NLA 104 begins with questions to determine which module an individual should undertake. Each assessment area is based on the latest scientific research and is presented based on identified risks and interests of the individual. Like the other assessments 100, the NLA 104 may be used to make lifestyle recommendations and guide an individual to a personalized intervention 110. The NLA 104 also helps direct an individual to other assessments 100, such as the genetic test 102 and/or biomarker test 106, which provide additional data to factor into an algorithm for selecting and administering a personalized intervention 110.

The NLA 104 may have about 250 to about 300 questions. The relevance of several questions is explained to the subject in, for example, a pop-up window. Some of questions are general and apply to all health conditions while others pertain to a specific health condition. For example, a program to support weight management may include inquiries into the types and/or amounts of foods eaten on a regular basis, the average calories consumed in a given period by the subject as well as the intensity and duration of activity the subject undertakes in a given period. For cardiovascular health, the questions may include those outlined in Table 1. Also included in Table 1 is the relevance information that may be presented in a pop-up window.

TABLE 1

| | |
|---|---|
| 1. | Do you know your total cholesterol or LDL cholesterol level? |
| | Total cholesterol level below 200 mg/dL is desirable. LDL-cholesterol below 130 mg/d is desirable and less than |
| | 100 mg/dL is considered optimal. |
| 2. | What is your IL-1 genotype? |
| | Individuals with ‘pattern 1’ IL-1 genotype have an increased risk of heart disease. |
| 3. | What is your CRP level? |
| | CRP is a marker of inflammation; elevated levels of this protein are emerging as a leading risk factor in heart |
| | disease. Elevated levels (>3 mg/dL) |
| 4. | Do you consume fewer than 2 servings of fish per week? |
| | The American Heart Association recommends eating two servings of fish per week to decrease your risk of heart |
| | disease. Cold water fish, such as salmon, tuna, mackerel, sardines and herring are the best source of omega-3 fatty |
| | acids that promote cardiovascular health. Increased fish intake helps to lower triglyceride levels, blood pressure and |
| | heart rate, and increase HDL-cholesterol levels; all of these changes are cardioprotective, and help to explain why |
| | increased fish intake lowers the risk of cardiovascular disease. |
| 5. | Do you drink several cups of green tea per day? Black tea? |
| | Did you know that specific foods, such as nuts, soy, legumes, tea, red wine, and garlic, have cardioprotective |
| | effects? The more of these types of foods you include in your diet, the more likely you are to have a healthy |
| | cardiovascular system. |
| 6. | Do you consume 1-2 glasses of wine (red) on a regular basis? |
| | Red wine is rich in antioxidants and cardioprotective phytonutrients such as quercetin and reseveratrol, and |
| | moderate consumption (1-2 glasses per day) is associated with a decreased risk of cardiovascular disease. The |
| | greatest benefit of drinking red wine comes when it is consumed with the meal. However, if you don't consume |
| | alcohol, this information should not encourage you to do so. |
| 7. | Do you consume 25 grams of soy protein per day? |
| | Regular consumption of soy protein (unlike protein from milk or meat), at a level of 25 g per day, in combination |
| | with a diet low in saturated fat and cholesterol may help to reduce the risk of coronary heart disease by helping to |
| | reduce cholesterol levels. |
| 8. | Do you consume 5 or more servings of fruits and vegetables each day? |
| | Did you know that simply changing your diet to include more fruits and vegetables can help to reduce your blood |
| | pressure? When a group of individuals increased their fruit and vegetable consumption by an average of 1.5 |
| | servings per day, their blood pressure readings decreased significantly. |
| 9. | Do you routinely eat salty foods or add salt to your food? |
| | The American Heart Association suggests that salt intake be limited to less than 6 grams per day (2,400 mg of |
| | sodium). However, researchers in the United Kingdom suggest an even lower intake of 3 grams per day, noting that |
| | greater reductions in salt intake dramatically reduces blood pressure, and this significantly reduces the risk of stroke |
| | and heart disease. |
| 10. | Do you exercise regularly? - i.e. 3-5 times per week, for at least 30 minutes per session? |
| | The benefits of physical activity are manifold, and especially important for the heart, as it improves heart function, |
| | lowers blood pressure and also lowers blood cholesterol. But how much exercise is enough? And at what intensity? |
| | It turns out that any amount of exercise is beneficial, and the more you exercise, and the greater the intensity, the |
| | greater the benefit (in general). In a study of older men (average 66 years), those who exercised with the greatest |
| | intensity, or expended greater than 1,000 calories per week had the lowest risk of coronary heart disease. A study of |
| | over 10,000 men and 3,000 women reveals that higher levels of physical fitness correlate to increased life |
| | expectancy, due to lower rates of cardiovascular disease and cancer. |
| 11. | Do you regularly consume low-dose aspirin? |
| | For individuals at high risk of heart disease, the American Heart Association recommends daily aspirin use (75-160 mg |
| | daily) to decrease risk. However, this recommendation does not apply to patients with aspirin intolerance (or |
| | allergy). It should also be noted that low-dose aspirin increases risk for gastrointestinal bleeding and hemorrhagic |
| | stroke, and should not be recommended in people at increased risk for these diseases. Benefits of reducing |
| | cardiovascular risk outweigh these risks in most patients with higher coronary risk. Doses of 75-160 mg per day are |
| | as effective as higher doses. |
| 12. | Do you supplement with folic acid at a level of 400 mcg/d (in a multivitamin, B complex, or |
| | Folate product)? |
| | A total of over 80,000 women were followed for 14 years to determine the relationship between heart disease and |
| | folate/B6 intake. The authors concluded that the risk of coronary heart disease is lowest in those women with the |
| | highest folate/B6 intake. This benefit is independent of source, meaning that both women with high dietary intake |
| | (non-supplement users) and those who took dietary supplements decreased their risk of heart disease. The benefit |
| | is graded, and corresponds to the amount of folate intake. Each 100 mcg/d increase in folate intake was associated |
| | with a 5.8% lower risk of coronary heart disease; although the benefit plateaus between 400-1000 mcg/d, and the |
| | benefits of supplementation above 1000 mcg/d were not examined. 9764 men and women in the US were followed |
| | for an average of 19 years to determine the relationship between folate intake and the incidence of stroke and |
| | cardiovascular disease. Individuals who consumed an average of 405 mcg folate per day had a 21% lower chance of |
| | a stroke than those who consumed an average of 99 mcg folate per day. Likewise, those with the higher folate |
| | intake had a 14% decreased risk of cardiovascular disease as well. |
| 13. | Are you currently on Hormone replacement therapy? |
| | Current research suggests that postmenopausal women on hormone replacement ttherapy (HRT) are at slightly |
| | elevated risk of coronary atherosclerosis, and have a slightly greater risk of dying from heart disease compared to |
| | women receiving a placebo. These latest findings are contrary to the original expectation of cardiovascular benefit, |
| | and indicate that postmenopausal women with coronary disease should be discouraged from HRT. |
| 14. | Do you currently supplement with beta-carotene? If so, how many mg per day? |
| | If you are a smoker, high dose, synthetic beta carotene supplementation (20-30 mg/day) is not recommended |
| | because it increases the risk of both cardiovascular disease and lung cancer. There is no indication that |
| | supplementation with low dose (4-6 mg/d) natural mixed carotenoids is harmful. On the contrary, limited |
| | laboratory scientific research at this point suggests that natural carotenoids may offer weak protection against lung |
| | damage induced by cigarette smoke in laboratory animals. |
| 15. | Do you have a strong social support network and spend time socializing with friends? |
| | A Swedish study of over 700 men involving 15 years of follow-up found that men who participated in the greatest |
| | amount of social and emotional contact reported significantly lower rates of heart disease. Men with the most social |
| | integration reduced their risk of heart disease by 55%, while those with the most emotional attachment reduced their |
| | risk by 42%. |
| 16. | Do you live in an urban environment, or an area subjected to excessive air pollution? |
| | Air pollution has detrimental effects not only on the respiratory system, but also on the heart, as it provokes |
| | inflammation, accelerates atherosclerosis and perturbs cardiac function. In fact, air pollution is twice as likely to |
| | cause death from heart disease as it is from respiratory ailments. As a result, individuals who live in large cities and |
| | polluted environments - from particulate matter emitted from cars, trucks, coal-fired plants and factories - are at an |
| | elevated risk of heart disease. |
| 17. | Do you have a family history (parents, grandparents, siblings) of heart disease? |
| | Even after other classic risk factors for heart disease have been taken into account, having a family history of heart |
| | disease significantly increases one's risk of heart disease. |
| 18. | Following exercise, does your heart rate reduce by more that 12 beats per min in the first |
| | minute post-exercise? |
| | All-cause mortality, and especially heart-related mortality is significantly higher in individuals who take a |
| | prolonged period to return to their resting heart rate following exercise. An abnormal heart rate recovery (defined as |
| | a reduction of 12 beats per minute or less in the first minute after exercise) is strongly predictive of death, increasing |
| | the relative risk by up to 4 times. |
| 19. | Do you have >3 of the symptoms of metabolic syndrome (Syndrome X) listed below? |
| | Increased waist circumference (>102 cm (40 inches) for men, >88 cm (36.5 inches) for women) |
| | Elevated triglycerides of 1.7 mmol/L (>150 mg/dl) |
| | Low HDL cholesterol (1.03 mmol/L (<40 mg/dl) for men; 1.29 mmol/L (<50 mg/dl) for women |
| | Hypertension: either systolic BP >130 mm Hg, or diastolic BP >85 mm Hg; or currently on antihypertensive |
| | medication. |
| | Impaired fasting glucose of 6.1 mmol/L (>108 mg/dl). |

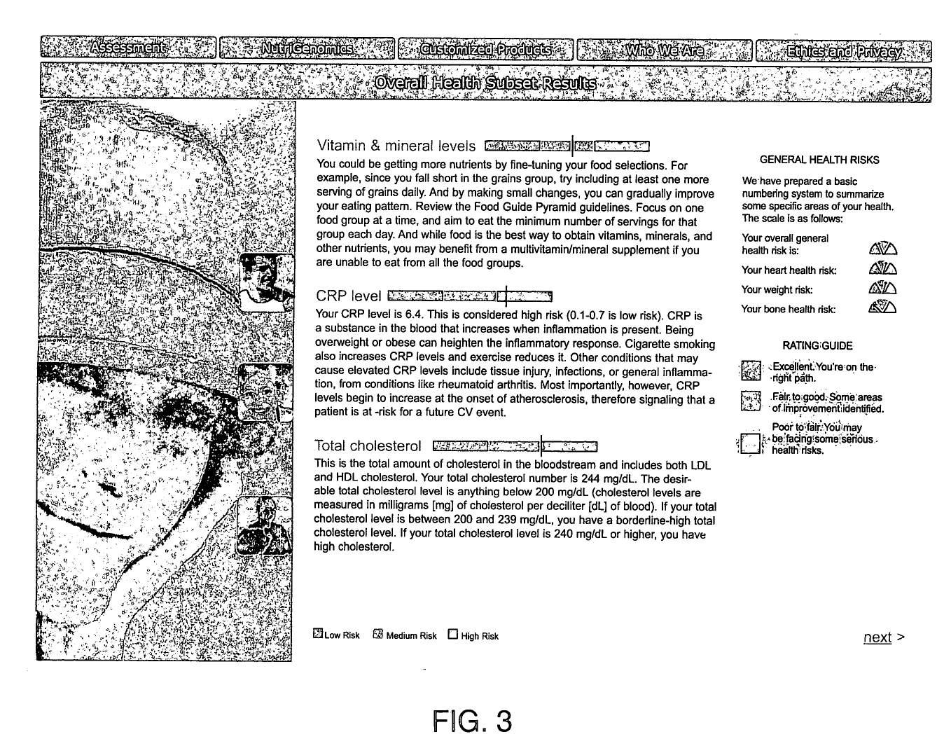

An individual's responses to the questions in the NLA 104, will be evaluated via algorithms, and a results and recommendation report 108 will be generated. The report 108 will advise personalized interventions 110 such as changes in behaviors or characteristics that are likely to improve one's health. The personalized interventions 110 may include lifestyle recommendations 310 and product recommendations or personalized compositions 300 to each individual to encourage them to start down the path toward optimal health. The algorithm employed to make the report 108 will be scientifically validated and supported by abundant scientific literature. Additionally, the report 108 may include information on or links to scientific websites and health and governmental agencies to provide scientific substantiation and rationale about the recommendations. As such, a significant educational element is embedded in the NLA 104 and report 108. FIG. 3 shows a sample results and recommendation report 108 for an individual who completed a lifestyle assessment 104 and a biomarker test 106. Individuals can take the NLA 104 multiple times and compare their health in different modules and in different time frames based on lifestyle modifications and biomarkers in order to measure improvement.

Another assessment tool is a genetic test 102 that helps determine an individual's predisposition or susceptibility to a health condition. Genetic makeup is increasingly being recognized as an important determinant of the impact of nutrition and lifestyle on risk for several health conditions including chronic degenerative diseases such as atherosclerosis, osteoporosis, rheumatoid arthritis. Duff G., Genetic Variation in Cytokines and Relevance to Disease in the Cytokine Network, Frontiers in Molecular Biology, 25; Balkwill F. (ed) Oxford University Press, March 2000; Chapter 7:152-173. While it is clear that reducing recognized risk factors for a particular health condition reduces risk in populations, individuals differ significantly in the degree to which these lifestyle and diet changes reduce risk. This is due, in part, to differences in genetic makeup, also known as genotype.

As part of the genetic test 102, the present invention includes a genetic test kit 170. This kit 170 is provided to individuals interested in a personalized intervention for a health condition that has been linked to a genotype. The genetic test kit 170 may include a non-invasive sample collection device such as a buccal swab or brush, container for protecting the DNA sample during transit to a testing lab, instructions for sample collection, an informational compact disc, and an informed consent agreement. Subjects will receive the genetic test kit 170 and collect biological samples containing DNA. The biological samples may include blood, urine, buccal cells, semen, skin cells, and hair. It is preferred that the collection device has a user performance specification that is equal to or better than less than 1 resample per 100 samples submitted. In this regard, a genetic test kit 170 might include multiple collection devices. Preferably, the container for shipping the DNA sample should conform to packaging and shipping regulations for biological samples.

To maintain confidentiality, the genetic test kit 170 contains unique identifier codes such that DNA samples cannot be readily linked to an individual. Random and unique identifiers include computerized bar codes, numerical codes, alpha codes, and alpha-numeric codes. The code may be placed on a perforated card or on a sticker that may be attached to the container containing the DNA sample. A copy of the code is retained by the subject to identify his/her lab results.

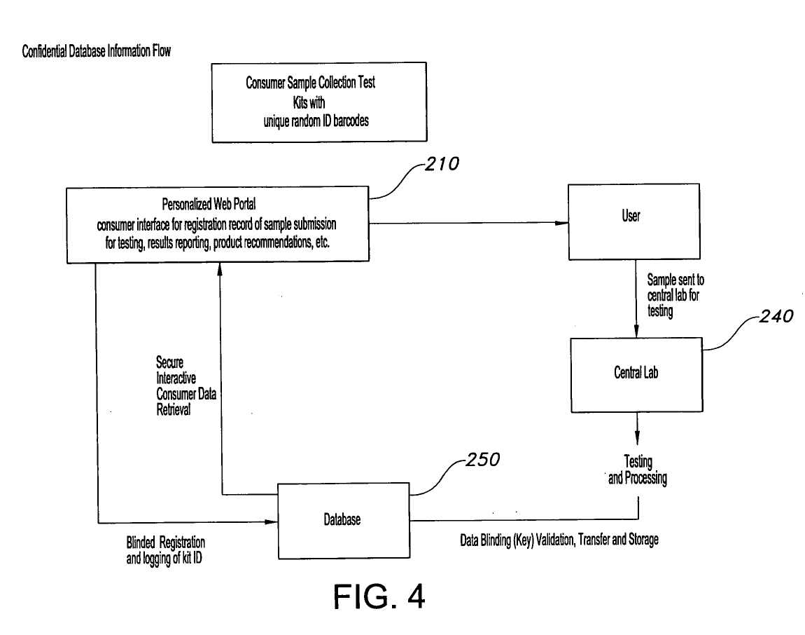

FIG. 4 shows one computer assisted embodiment of the confidential information flow using a unique identifier code. In FIG. 4, information is being collected and conveyed via a personalized health web portal 210. Once a subject completes the DNA sample collection process, the sample is sent in for analysis in a testing laboratory 240. The testing laboratory 240 provides the results of the test, using this unique identifier code, to a secure HIPAA & PIPED third party web server or database 250. The database 250 supports the personalized web portal 210 and may house algorithm programs that drive the results report 108 having the personalized intervention 110 recommendations. The algorithm program in the database 250 may include the same algorithm that is used to generate the personalized intervention 110 recommendation based on the NLA 104. When retrieving results of the genetic test 102, an individual may need to access their personalized web portal 210 and provide their unique identifier code. This identifier code may then be linked to the corresponding lab results in the third party web server or database 250. All results reports 108 will be viewable on the personalized web portal 210. The database 250 is thus used for receiving, storing, and/or sending information related to the genetic test 102 or other assessments 100. The database 250 may receive and track health information input by service laboratories and individuals such as biomarker, genotype, and Framingham data (www.framingham.com/health). The database 250 may also be directly accessible for research purposes (de-identified data) by appropriately qualified research staff. In this capacity, the database 250 may function as a registry database for tracking health status in individuals of known genotype.

In one embodiment of the present invention, a genetic test 102 for regulating inflammatory conditions is provided. As such, the genetic test 102 may measure variations in the Interleukin-1 (“IL-1”) gene cluster and assign subjects to a predetermined inflammatory genotype or genetic pattern which is associated with a health condition and a personalized intervention 110. A strengthening body of data suggests that inflammation as indicated by increased IL-1, tumor necrosis factor alpha, interleukin-6, and elevated acute phase proteins such as fibrinogen and C-Reactive Protein (“CRP”), is common to many chronic degenerative diseases, such as heart disease. IL-1, a key cytokine regulator of the inflammatory response, has emerged as playing a particularly important role at the genetic level in determining the degree to which the inflammation pathway is turned on. IL-1 is a general name for two distinct proteins, IL-1 alpha and IL-1 beta, that are considered the first of a small, but possibly growing, family of regulatory and inflammatory cytokines. Along with IL-1 receptor antagonist and IL-18, these molecules play important roles in the up and down regulation of acute inflammation. In the immune system, the production of IL-1 is typically induced, generally resulting in inflammation. The strong influence of IL-1 over the inflammation pathway follows from its functional role as one of the initiating cytokine signals in the inflammatory pathway.

Recent research has identified polymorphisms in the IL-1 gene that lead to over expression or altered biological activity of IL-1 and elevated levels of the inflammation biomarker, such as CRP. Berger P et al., CRP levels are influenced by common IL-1 gene variations; Cytokine 17:171-174 (2002). Individuals with selected polymorphisms associated with over expression and under expression of IL-1 appear to be at increased risk for selected chronic degenerative diseases. The mechanistic role of IL-1 in the overall inflammatory response and the detrimental impact of IL-1 over expression thus creates a need to address an individual's risk for inflammation, followed up with an IL-1 genotype directed intervention. U.S. Pat. Nos. 6,268,142; 6,210,877; and 6,524,795 discuss gene IL-1 polymorphisms in greater detail and are incorporated in their entirety by reference.

For example, for osteoporosis and cardiovascular disease, there are three patterns as outlined in Table 2: pattern 1 (including sub-patterns A, A/B, and B), pattern 2, and pattern 3. These three patterns are determined by detecting particular IL-1 genotype polymorphisms located on one or more of the following IL-1 genes and positions: IL-1A (+4845), IL-1B (+3954), IL-1B (−511), and IL-1RN (+2018). For example, pattern 1 includes individuals with the following allelic pattern: allele 2 on IL-1A (+4845), allele 2 on IL-1B (+3954), and allele 1 on IL-1B (−511). Pattern 1 indicates that the subject has a predisposition to increased levels of inflammation and should periodically monitor his/her biomarker of inflammation, CRP, to ensure it is within the normal range. In addition, a pattern 1 individual may consider mitigating his/her inflammatory response through the personalized intervention 110 based on results from one or more assessments 100. Pattern 2 includes individuals with the following allelic pattern: allele 1 on IL-1A (+4845), allele 1 on IL-1B (+3954), and allele 2 on IL-1B (−511). Pattern 2 indicates that the subject has a predisposition to increased levels of cholesterol and should periodically monitor his/her cholesterol levels. The subject should also follow the personalized intervention 110 recommendation. Pattern 3 includes individuals with the following allelic pattern: allele 1 on IL-1A (+4945), allele 1 on IL-1B (+3954), and allele 1 on IL-1B (−511). Pattern 3 indicates that the subject is not predisposed to either increased levels of inflammation or cholesterol. However, based on the subject's lifestyle, lifestage, and nutritional intake, the subject may still be recommended to periodically check his/her biomarker levels.

TABLE 2
Genetic Interpretation for Cardiovascular Disease & Osteoporosis Product
Applications

| Genetic | | | | | |
|---|---|---|---|---|---|
| Pattern | 1A | 1AB | 1B | 2 | 3 |
| % of | 4 | 33 | 11 | 31 | 21 |
| Population* | | | | | |
| IL-1 | HIGH | HIGH | MID-RANGE | LOW | MID- |
| Expression | | | | | RANGE |
| Health | 3 to 4 times | 3 to 4 times | Predisposition | High risk | Least risk |
| Issues | greater risk | greater risk of | for | factors for | for MI, |
| | of | MI. | osteoporotic | coronary | osteoporosis |
| | myocardial | Predisposition | vertebral | artery | and |
| | infarction | for | fractures | stenosis | stenosis. |
| | (MI) | osteoporosis | | | |

*Individuals of Western European descent

An individual may also choose to utilize a biomarker test 106 as one assessment 100 if a biomarker is associated with the health condition at issue. While the genetic test 102 assists in determining the subject's susceptibility to a health condition, the biomarker test 106 assists in assessing the subject's current state of wellness or illness with respect to the health condition. Like the other assessments 100, the biomarker test 106 will assist in selecting a personalized intervention 110. Further details on the biomarker test 106 are provided in the “Monitoring” section presented below.

It is envisioned that the results report 108 of one assessment 100 will provide instantaneous personalized intervention 110 recommendations upon completion. However, the gold tier 150 or the NLA 104 in combination with the genetic test 102 and the biomarker test 106 provides the most comprehensive personalized intervention 110 recommendation.

Education and Counseling

The present invention may also include education and training 140, links or access to medical professionals, and coaching/counseling 130. This aspect of the invention provides individuals with health information required to change and sustain positive behaviors. Educational solutions may take many forms. For example, the education may be embedded in the NLA 104. Additionally, a learning center 180 may be provided as a link on the personalized web portal 210 so that individuals have access to a collection of health related information. The learning center 180 includes self-paced health education and personalized health webinars and other on-line learning tools to better equip individuals with the information they need to be successful in the program. Other educational tools are video presentations of the program, white papers on health topics, links to external resources, FAQ'S, nutrient reference desk, and a glossary of nutrigenomic and dermigenomic terms.

For the coaching and counseling 130 aspect of the invention, a confidential third party service may be provided to give personalized feedback, advice, and guidance so individuals can achieve their health goals. To facilitate this aspect, the website 200 may include contact information, chat rooms, and links to coaches and counselors for particular health issues. In a preferred embodiment, coaching and counseling 130 is set up under four levels of support. Table 3 outlines this aspect of the invention in further detail.

TABLE 3
LEVEL I - Customer Care Expert

| Type of information/questions: | Role: Personalized Health Program Expert |
|---|---|
| How much does the program | Understands basic feature and benefits of the program |
| cost? | Knows how to direct calls to the appropriate customer care |
| What are the different levels of | service |
| the program? | Where to go for more information |

LEVEL II - Coaching

| | Role: Health care professionals (have an in-depth |
|---|---|
| Type of information/questions. | understanding of health) |
| All of the above PLUS: | Advanced understanding of program features and benefits. |
| How do I stay motivated to | Why is this different and what is the value. |
| continue the recommended | How to overcome objections. |
| intervention? | Understands - supplementation, the health industry, diet |
| How do I work exercise into | and nutritional needs. |
| my lifestyle? | Able to facilitate and guide individual through program |
| How much vitamin C should I | Provide information on general health risks, behavior |
| take? | change and goal setting. |

LEVEL III - Consultation

| | Role: Certified health care providers (Nurse, dietician, |
|---|---|
| Type of information/questions | doctor) |
| What do my results mean? | Ability to interpret results from a medical standpoint as |
| | well as a program standpoint. |
| | Understands the importance of supplementation, diet and |
| | exercise. |

LEVEL IV - Counseling

| | Role: Health care provider with experience and/or |
|---|---|
| Type of information/questions: | specialization in genetic counseling |
| What should I do with this | Ability to counsel individuals on their predisposition to |
| genetic information? Call | health conditions. |
| family? Tell my children? | Ability to empathize, de-escalate and talk through the |
| An individual is panicking due | impact of their results. |
| to the results. | |

Intervention

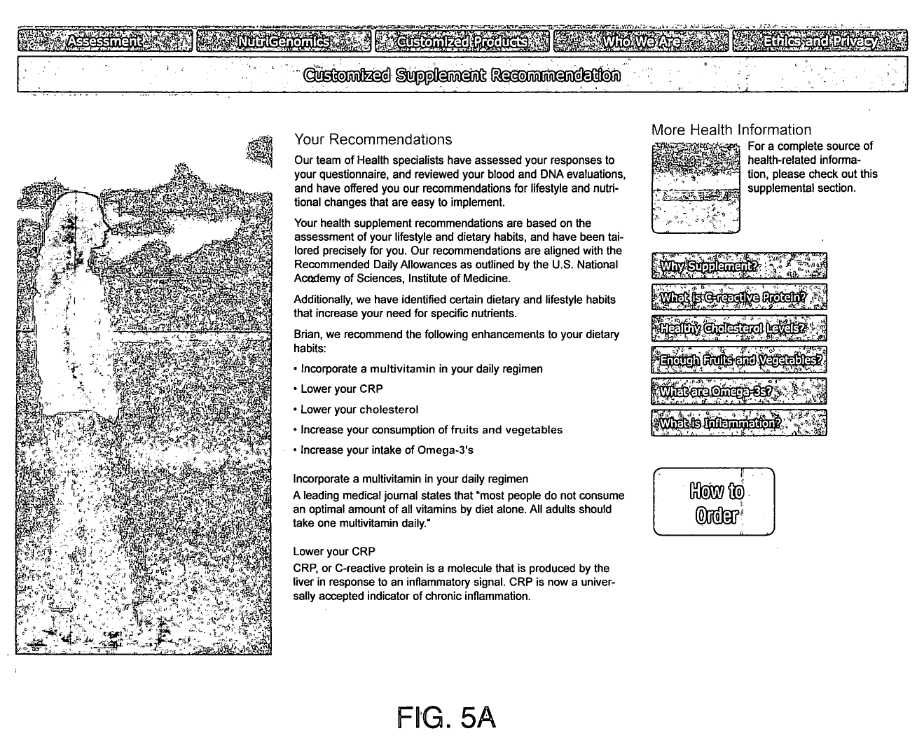

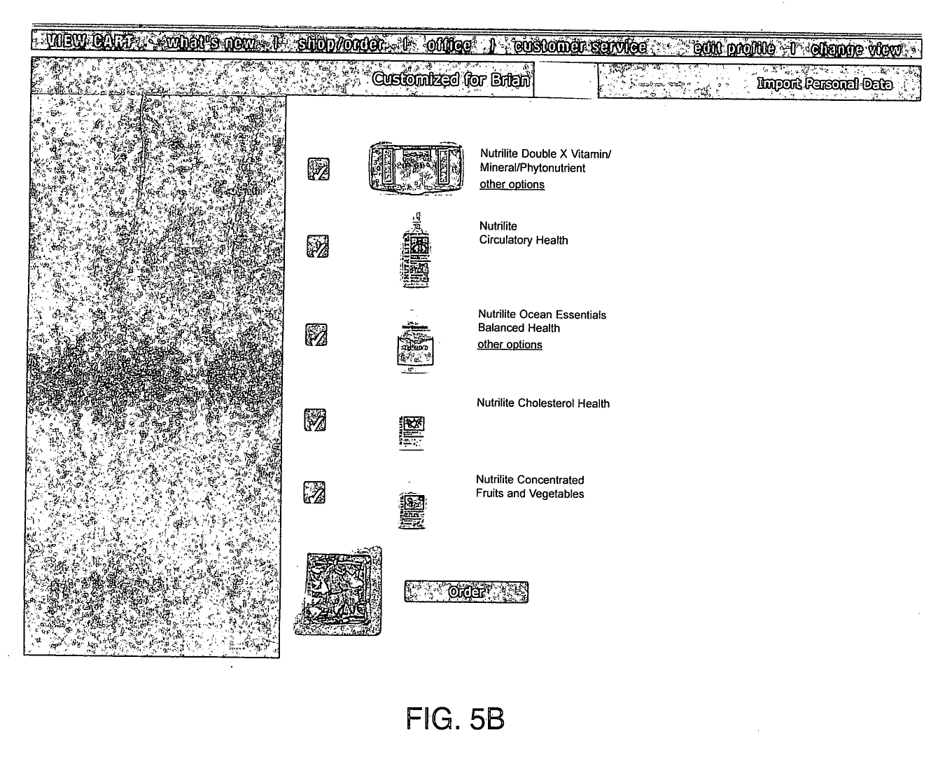

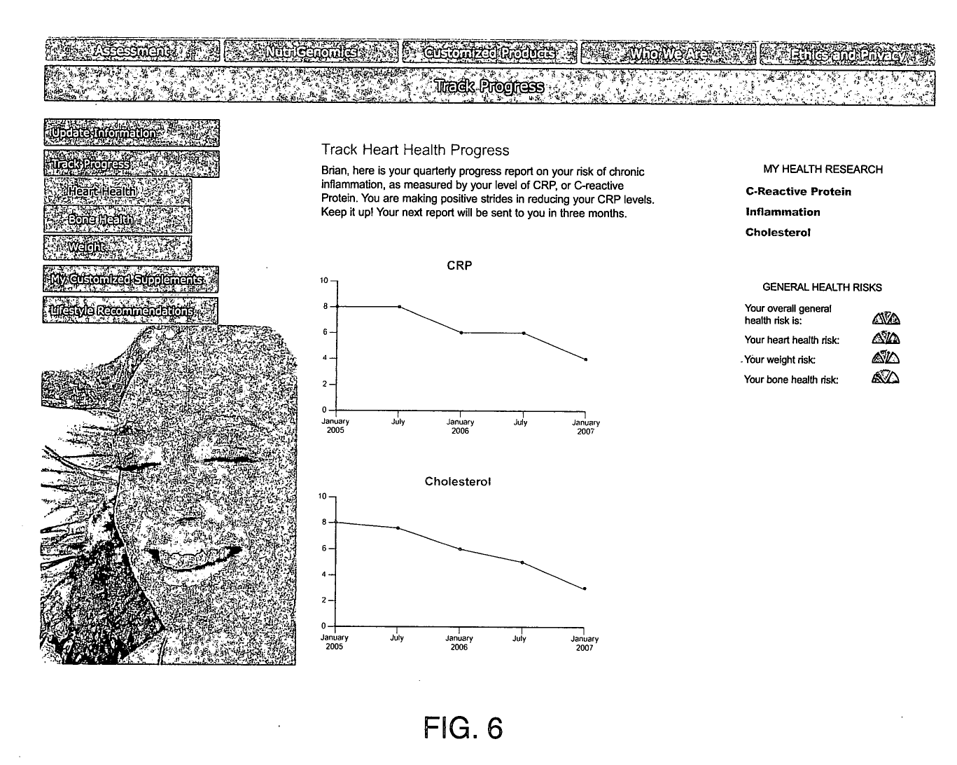

As a result of identified health conditions and health interests of the individual, the present invention includes providing a personalized intervention 110 to regulate the health condition identified in the assessments 100. FIGS. 5A and 5B show a sample web page outlining a personalized intervention 110 recommendation. It is contemplated that the personalized intervention 110 may include a personalized composition 300, a lifestyle recommendation 310, or a combination of both.

Lifestyle recommendations 310 include fitness programs and weight management/loss interventions to support weight management, increase muscle density, and/or endurance. For example, a lifestyle recommendation 310 is generated from the user's current lifestyle and the health risks identified in the NLA 104. The plan will detail recommended changes, such as trimming fats or increasing cardiovascular workouts, and provide potential benefits described through decreased risks.

Personalized compositions 300 may include a single product, such as a dietary supplement or lotion that has been formulated with specific ingredients based on responses to one or more of the health assessments 100. Personalized compositions 300 may also include several products that satisfy the health needs of the individual. Personalized compositions 300 may be in any form, but oral and topical forms are preferred because of their convenience. Non-limiting delivery forms for personalized compositions 300 for the present invention are nutritional supplements, drinks, drink mixes, foods, creams, lotions, ointments, emulsions, powders, and transdermal patches. The results report 108 will identify personalized compositions 300 that may improve an individual's health condition. The results report 108 will also provide recommended levels of the personalized compositions 300.

In one embodiment, the present invention includes a personalized composition 300 developed to regulate inflammatory conditions. In this embodiment, the personalized composition 300 may be targeted toward regulating the over-expression of IL-1 in key tissue areas, most notably heart and bone tissue. Specifically, the personalized composition 300 is intended to regulate the over-expression of IL-1 genes associated with osteoporosis (pattern 1AB & 1B) and cardiovascular disease (pattern 1A & 1AB) risk. The personalized composition 300 for osteoporosis is expected to be different from the personalized composition 300 for cardiovascular disease due to the fact that IL-1 in different tissue or bone cells will respond differently to nutritional ingredients. Therapeutics to regulate the under-expression of IL-1 genes associated with stenosis (pattern 2) as well as therapeutics to maintain healthy levels of IL-1 expression (pattern 3) are also envisioned. It is preferred that a measurable change in biomarkers is evident within 3 months of administering the personalized composition 300.

Ingredients that are efficacious on regulating IL-1, thereby reducing or eliminating an immunomodulatory and/or inflammatory response are identified in U.S. Application No. 60/502,755 which is incorporated in its entirety by reference. It is believed that the IL-1 therapeutic compositions will still provide benefits to those that are not responsive to biomarker reduction. This is due to the fact that, while there are other factors downstream of IL-1 which can affect the biomarkers, this does not necessarily negate the effects of regulating IL-1 upstream because it affects many downstream pathways and confers many benefits.

In addition to any personalized intervention 110, Table 4 provides an exemplary list of nutritional products that may be recommended to an individual to further support the health condition identified through the assessments 100. These nutritional products are manufactured by Access Business Group LLC, Ada, Michigan.

TABLE 4

| Nutritional Products | Structure/Function |
|---|---|
| Fruit & Vegetable supplement (lycopene, | Lifestyle habits (smoker/bad environment, diet) |
| lutein, quercitin, ellagic acid, hesperidin, | |
| EGCG) | |
| Glucosamine | Overweight, Joint mobility, Pattern 1A or |
| | elevated risk for CVD, elevated CRP |
| Vitamin C | Lifestyle habits (smoker/bad environment, diet) |
| Coenzyme Q10 | Heart |
| Saw Palmetto | Prostate |
| Ginseng | Fatigue |
| Antioxidants | Lifestyle habits (smoker/bad environment, diet) |
| | Work-out frequently |
| Bilberry w/lutein | Vision |
| Parselenium E | Brain, Heart |
| Omega-3 | Heart, Joints, Pregnancy, Poor Diet |
| Calcium and magnesium | Osteoporosis, Bone Health |
| Green tea extract | Heart |
| Vitamin B | Heart/homocysteine |
| Ginkgo biloba w/dha | Brain |
| Garlic herbal | Heart-multifactorial |
| Digestive enzymes (proteolytics, | Digestion |
| carbohydrolytics, and lipolytics) | |
| Biotin, Collagen | Hair, skin, nails |
| Chromium picolinate | Blood glucose regulation |
| Black cohosh | Women with hotflashes |
| Milk thistle | Toxic liver exposure - alcohol, acetaminophen |
| Ipriflavone | Bone |
| Mushroom extract | Immune System |
| Primrose plus | Pre-Menstrual Syndrome |
| Folic iron | Childbearing/lactating |
| St. John's wort | Mild depression |
| Multicarotene | Skin and Eyes |
| Multivitamin | Energy/general health |

The program may include an option for delivery of a personalized composition 300 in a custom packet in combination with a variety of other personalized therapeutic compositions. For example, one packet may contain eight different dietary supplements. The customized packets may be personalized with labels having the subject's name and contain personalized compositions 300 recommended from the subject's results reports 108. The compositions 300 can be packaged in a single administration packet, pouch, envelope, or other container. As such, an individual has all the products he/she needs for a single administration in one convenient packet. A monthly supply of these packets may be provided to the subject. It is believed that the single monthly serving size as well as the convenient, portable packets associated with the customized products will encourage a pattern of regular use.

Monitoring

Monitoring 120 the effect of the personalized intervention 110 is another aspect of the present invention. An individual will be able to monitor his or her health condition by undergoing a follow-up NLA 122 or a follow-up biomarker test 124 that confirms that the intervention 110 is regulating the health condition. Follow-up biomarker tests 124 for a fitness program to increase muscle density may include monitoring 120 muscle size, body fat, waist to hip ratio, and weight, while programs for regulating a disease may include a test for blood pressure and heart rate.

Biomarkers are specific physical characteristics used to measure some of the complex chemical changes in the body that lead to disease. This measurement is especially useful for chronic diseases and health conditions where the chemical changes start many years before the disease is evident. As mentioned in the section titled “Assessments” above, a biomarker test 106 can be performed before an intervention 110 is administered to obtain a baseline reading of health. Additionally, it can be performed to monitor progress following intervention 110. Preferably, the follow-up biomarker test 124 is performed on individuals who remain on the personalized intervention 110 and measured about 6 months after the initial adminstration of the personalized intervention 110. The follow-up biomarker test 124 measures a chemical change for a specific analyte that has been associated with risk for a specific disease and provides a tool to guide individuals toward personalized interventions 110.

As part of the biomarker tests 106 and 124, the program includes a biomarker test kit 172. The kit 172 may contain a biomarker collection device, instructions, information compact disc, alcohol swabs, bandages, and informed consent forms. To maintain confidentiality, the kits 172 may contain unique identifier codes such that a biomarker sample cannot be readily linked to an individual. The coding of the biomarker kits 172 can be accomplished in the same manner as the genetic test kits 170 mentioned above.

It is preferred that the collection device is non-invasive or minimally invasive. The collection device may include a minimally invasive lancet and a blood spot collection card/paper. The BD Genie™ lancet is an acceptable lancet and can be obtained from Becton Dickinson of Franklin Lakes, N.J. An individual may use the lancet device at home to produce a drop of blood, which is then collected on collection paper and sent to a testing laboratory 240 to analyze the biomarkers. Preferably the collection paper is a 903™ blood spot card from Schleicher and Schuell, Keene, N.H. Although the biomarker testing laboratory and the genetic testing laboratory are represented by a single box 240 in FIG. 2B, different laboratories can be utilized. At present, most biomarkers are routinely measured in blood. To make biomarkers available routinely to a broader group of individuals, the present invention envisions tests for biomarkers using samples collected either by a special mouth swab or tape applied to the skin. It is preferable that the performance specification for the unskilled user is less than 1 resample/100 samples submitted. In a preferred embodiment, the biomarker test kit 172 is packaged or bundled with a genetic test kit 170.

Once a subject collects a biological sample containing a biomarker, the subject sends the biomarker sample to a testing laboratory for analysis. Once complete, the lab will input the user's biomarker data to a confidential database and will notify the individual that his/her test results are ready to view. The results may be reported in a simple and easy to understand format. The user will need to use his or her identifier code to retrieve the biomarker test results, preferably from a personalized web portal 210. It is envisioned that there will be detectable and meaningful changes in biomarkers measured by tests within three months of administering the personalized composition 300. Biomarker tests 106 and 124 enable a subject and/or healthcare professional to monitor the impact of a supplement product and/or lifestyle changes on a known biomarker that has been correlated with disease risk. A number of biomarkers have been identified for certain disease and are contemplated for the present invention.

For some chronic diseases, the biomarkers are well-defined such as a CRP and cholesterol for heart health. CRP is a plasma protein within the bloodstream that is increased during an inflammatory process. CRP has been used for many years as a marker of inflammation and is one of the more specific markers of risk. An individual with CRP that is chronically above a certain level is known to be at an increased risk for future heart attacks as well as other chronic diseases. For example, when CRP is elevated in the baseline state, the risk of developing atherosclerotic vascular disease is anywhere from 3-6 times higher than the average population. Another heart health biomarker includes cholesterol. Cholesterol is a fatty substance that is an important part of the outer lining (membrane) of cells. Cholesterol is carried in the bloodstream as lipoproteins. Low-density lipoprotein (LDL) cholesterol is the “bad” cholesterol because elevated LDL levels are associated with an increased risk of coronary artery (heart) disease. Conversely, high-density lipoprotein (HDL) cholesterol is the “good” cholesterol since high HDL levels are associated with less coronary disease.

Biomarkers for osteoporosis include one or more bone biomarkers of resorption, such as pyridinium cross-links of colllagen and the amino- and carboxy-terminal telopeptides of these cross-links and one or more biomarkers of bone formation, such as bone specific alkaline phosphatase (BAP), precollagen extension pepetides, and osteocalcin. Biomarkers for weight management may include blood sugar, insulin, triglycerides, and free fatty acids. Biomarkers for other health conditions such as Alzheimer's disease and premature skin wrinkling may not be as well defined.

Some of the biomarkers, such as CRP, have already been shown to be lowered by specific nutrients. The use of biomarkers in combination with genetic tests 102 appear to offer great potential to extend wellness by guiding development and targeting use of nutritional supplements, skin care products, and other interventions. Table 5 is an example of the type of conclusions that can be drawn from subjects that undergo both a genetic test 102 for inflammation and a CRP biomarker test.

TABLE 5
Biomarker Test Results & Interpretation - CVD & Pattern 1

| | | BIOMARKER TEST | |
|---|---|---|---|
| | | Elevated/Positive | Normal/Negative |
| GENETIC TEST | Pattern 1 | A | B |
| | Present or | Life-long genetic tendency | Life-long genetic tendency |
| | Expressed | to excess inflammation | to excess inflammation |
| | | Already showing signs of | Not yet showing signs of |
| | | excess inflammation | excess inflammation |
| | | Recommend intervention | Recommend intervention |
| | | to reduce inflammation | to assist in maintaining low |
| | | About 34% of population* | inflammation |
| | | | About 3% of population* |
| | Pattern 1 | C | D |
| | Absent | Does not have a genetic | Does not have a genetic |
| | | tendency to show excess | tendency to excess |
| | | inflammation | inflammation |
| | | Showing signs of excess | Not showing signs of |
| | | inflammation due to other | excess inflammation due to |
| | | factors | other factors |
| | | Recommend intervention | Maintain correct activities |
| | | to reduce inflammation | and actions & recommend |
| | | About 47% of population* | checking biomarker again |
| | | | in 1 to 2 years |
| | | | About 16% of population* |

*Individuals of Western European descent

To support the monitoring 120 aspect of the invention, tracking tools 260 are provided. The input of biomarker data (such as cholesterol levels and CRP) as well as lifestyle and diet information into the tracking tools 260 database will provide a baseline trend for certain health risks, such as cardiovascular disease, osteoporosis, and obesity. This trend will convey the degree of risk associated with each area of health. The input of additional biomarker results and lifestyle changes into the tracking system will allow the user to see improvement in risk areas. The tracking tools 260 also include a risk scenario generator 262 to hypothesize mitigation or risk increase based on potential future improvements or regressions. Pictures and graphs depicting the affect of certain behaviors on health are provided for the user. For example, for osteoporosis, a picture of a healthy individual may be shown next to an individual who's posture has been affected by poor exercise and eating habits.

It is to be understood that the foregoing specification of this invention is illustrative and has been described in relation to certain preferred embodiments. It will be apparent to those skilled in the art that the invention is susceptible to alteration and that certain other details described herein can vary considerably without departing from the basic principles of the invention as defined in the following claims.

1. A program for regulating health conditions in a subject comprising:
(a) providing a genetic test for detecting an inflammatory genotype;
(b) assessing whether the subject is susceptible to a health condition based on the inflammatory genotype;
(c) administering a personalized composition to the subject for regulating the health condition associated with the inflammatory genotype;
(d) monitoring the health condition of the subject to determine the subject's response to the personalized composition, wherein the monitoring step occurs after the administering step and includes measuring a biomarker having a correlation to the health condition.

2. The program of claim 1 wherein the inflammatory genotype is an IL-1 genotype.

3. The program of claim 1 wherein the health condition is selected from the group consisting of cardiovascular disease, osteoporosis, weight management, obesity, skin related conditions, and hair related conditions.

4. The program of claim 1 wherein the biomarker is selected from the group consisting of: CRP, cholesterol, and biochemical markers of bone turnover.

5. The program of claim 1 wherein the personalized composition is packaged in single administration packets and includes personalized labels.

6. The program of claim 1 wherein the genetic test includes a genetic test kit having a unique identifier code.

7. The program of claim 1 wherein the genetic test includes a genetic test kit having a DNA collection device and a container for shipping a DNA specimen collected on the DNA collection device.

8. The program of claim 1 further comprising providing a web portal for receiving and sending data to facilitate any one of steps (a)-(d), wherein the web portal is accessible by a unique identifier code.

9. The program of claim 1 wherein the monitoring includes a computer assisted tracking program for providing historical testing information.

10. The program of claim 1 further comprising providing a biomarker test before assessing whether the subject is susceptible to a health condition based on the inflammatory genotype.

11. The program of claim 1 further comprising providing a lifestyle assessment and a biomarker test before assessing whether the subject is susceptible to a health condition based on the inflammatory genotype.

12. The program of claim 1 further comprising storing one or more of the genetic results, the personalized composition, or the subject's response to the personalized composition on a computer, a personal storage device, or both.

13. The program of claim 12 wherein the subject can obtain the stored information through a secure access to the computer or personal storage device.

14. The program of claim 1 further comprising providing computer assisted counseling.

15. The program of claim 1 further comprising providing computer assisted education on nutrigenomics.

16. The program of claim 1 further comprising providing an on-line lifestyle assessment wherein the assessment includes questions associated with at least one pop-up window having information on the question's relevance to the health condition.

17. The program of claim 1 further comprising developing the personalized composition based on the assessment.

18. The program of claim 1 wherein the monitoring the health condition is periodic.

19. The program of claim 1 further comprising assessing changes based on the monitoring the health condition.

20. The program of claim 1 further comprising sending targeted messages to a subject based on the genetic test, the subject's response to the personalized composition, or both.

21. The program of claim 1 wherein one or more steps is performed using a system of networked computers that include one or more of software for organization of database information, secure transactions, or web browser readable documents and forms.

22. A method of assessing and monitoring cardiovascular health comprising:
(a) providing a genetic test for detecting the presence of an IL-1 inflammatory genotype;
(b) providing a personalized composition for modulating an IL-1 inflammatory genotype expression; and
(c) providing a CRP biomarker test for monitoring cardiovascular health subsequent to providing a personalized composition;

23. The method of claim 22 wherein the personalized composition comprises at least one of rosehips, nettle root, olive extract, blackberry, blueberry, elderberry, Afromomum melegueta, and resveratrol.

24. The method of claim 22 wherein the IL-1 inflammatory genotype comprises one or more of the following polymorphisms: IL-1A (+4845), IL-1B (+3954), IL-1B (−511), and IL-1RN (+2018).

25. The method of claim 22 further comprising repeating steps (b) and (c).

26. A computer assisted method for regulating health conditions in a subject comprising:
(a) providing a genetic test for detecting an IL-1 genotype;
(b) providing a lifestyle assessment;
(c) providing a biomarker test for measuring CRP;
(d) assessing whether the subject is susceptible to a health condition based on the IL-1 genotype and based on results from the lifestyle assessment and the biomarker test;
(e) providing a personalized dietary supplement to the subject based on the health condition identified in any one of steps (a)-(c);
(f) monitoring the health condition of a subject to determine the subject's response to the personalized dietary supplement, wherein the monitoring step occurs after providing the personalized dietary supplement and includes a follow-up biomarker test and a tracking tool for displaying the results of the biomarker test and the follow-up biomarker tests;
(g) providing education and counseling services to the subject; and
(h) providing a personalized web portal for sending and receiving information for facilitating any portion of steps (a)-(g).

27. The program of claim 26 further comprising sending targeted messages to a subject based on the genetic test, the biomarker test, the lifestyle assessment, the subject's response to the personalized dietary supplement, or the follow-up biomarker test.

28. A computer assisted method for regulating health conditions in a subject comprising:
(a) providing a first dataset on a data processing apparatus where the first dataset comprises information that correlates a unique identifier code for the subject with a genetic test result;
(b) providing a second dataset on a data processing apparatus where the second dataset comprises information that correlates a personalized composition with one of the subject or the unique identifier code;
(c) providing a third dataset on a data processing apparatus where the third dataset comprises information that correlates the subject's response to the personalized composition with one of the subject or the unique identifier code;
(d) preparing a report containing information for one of the genetic test result, the personalized composition, or the subject's response to the personalized composition.

29. The method of claim 28 wherein the report is accessible to the subject by the unique identifier code.

30. A computer apparatus for use in regulating health conditions in a subject programmed to send and receive genetic test results, send and receive information on a personalized composition, send and receive the subject's response to the personalized composition.
